(19) United States
(12) Patent Application Publication
(10) Pub. No.: US 2021/0170044 A1
(43) Pub. Date: Jun. 10, 2021

(54) SYSTEMS AND METHODS FOR LYSOSOME INDUCED IMMUNOGENIC CELL DEATH

(71) Applicant: Antigenesis LLC, Cambridge, MA (US)

(72) Inventor: Eric T. Fossel, Cambridge, MA (US)

(73) Assignee: Antigenesis LLC, Cambridge, MA (US)

(21) Appl. No.: 16/762,899

(22) PCT Filed: Nov. 15, 2018
(86) PCT No.: PCT/US2018/061304

§ 371 Date: May 8, 2020

Related U.S. Application Data
(60) Provisional application No. 62/740,477, filed on Oct. 3, 2018.
(60) Provisional application No. 62/587,207, filed on Nov. 16, 2017.

(51) Int. Cl. A61K 47/68; A61K 38/44; A61K 31/20; A61K 31/40; A61P 35/00

(52) U.S. Cl. CPC A61K 47/6851; A61K 47/6815; A61K 38/443; A61P 35/00; A61K 31/20; A61K 31/40; A61K 38/44

(57) ABSTRACT

The present invention generally relates to systems and methods for treating cancer using immunogenic cell death, e.g., lysosome-induced immunogenic cell death. This includes at least three aspects, which may be used separately or together. A first aspect involves the preparation of a tumor for treatment, for example, by withdrawal and suppression of antioxidants, supply of n-3 through n-6 and other unsaturated fatty acids, and/or treatment of the subject with statins. A second aspect involves restoration of p53 functionality though genetic manipulation, including techniques involving CRISPR. A third aspect involves constructing an antibody-enzyme complex where the antibody recognizes the tumor and the enzyme is an oxidase. The complex may be administered to a subject. The subject may also be provided with a substrate to the enzyme. These and/or other aspects, may be used separately or together, and may be used to treat and/or cure cancer. In some cases, the lysosome may be targeted in inducing cell death, e.g., in cancer cells.

### RELATED APPLICATIONS

This application claims the benefit of U.S. Provisional Patent Application Ser. No. 62/587,207, filed Nov. 16, 2017, entitled “Lysosome initiated Apoptiosis—A method for cancer therapy,” by E. Fossel; and of U.S. Provisional Patent Application Ser. No. 62/740,477, filed Oct. 3, 2018, entitled “Systems and Methods for Initiating Apoptosis to Treat Cancer,” by Fossel. Each of these is incorporated herein by reference in its entirety.

### FIELD

The present invention generally relates to systems and methods for treating cancer using immunogenic cell death. For example, it is the purpose of certain embodiments of the invention to treat and/or cure cancer by targeting the lysosome, inducing its membrane to become permeable and leak out degradative enzymes, resulting in digestion of the inside of the cell, resulting in apoptosis, thus causing lysosome-induced immunogenic cell death.

### BACKGROUND

Cancer is a group of diseases involving abnormal cell growth, with the potential to invade or spread to other parts of the body. While cancers can be treated with techniques such as radiation therapy, surgery, chemotherapy, or targeted therapy, new techniques are still needed, given the relatively high mortality rate of cancer victims.

### SUMMARY

The present invention generally relates to systems and methods for treating cancer using immunogenic cell death, e.g., lysosome-induced immunogenic cell death. The subject matter of the present invention involves, in some cases, interrelated products, alternative solutions to a particular problem, and/or a plurality of different uses of one or more systems and/or articles.

In one aspect, the present invention is generally directed to an article. In one set of embodiments, the article comprises a complex comprising an antibody that recognizes a tumor, and an enzyme that is able to increase lysosomal membrane permeability. In another set of embodiments, the article comprises a complex comprising an antibody that recognizes a tumor, and an enzyme that is able to create reactive oxygen species.

The present invention, in another aspect, is generally directed to a method. In one set of embodiments, the method comprises administering, to a subject, a composition comprising a complex of an antibody that recognizes a tumor, and an enzyme that is able to increase lysosomal membrane permeability. According to another set of embodiments, the method comprises administering, to a subject, a composition comprising a complex of an antibody that recognizes a tumor, and an enzyme that is able to create reactive oxygen species. The method, in yet another set of embodiments, comprises administering, to a subject, a composition comprising a complex of an antibody that recognizes a tumor, and an enzyme that is able to cause lysosomal leakage.

Several methods are disclosed herein of administering a subject with a compound for prevention or treatment of a particular condition. It is to be understood that in each such aspect of the invention, the invention specifically includes, also, the compound for use in the treatment or prevention of that particular condition, as well as use of the compound for the manufacture of a medicament for the treatment or prevention of that particular condition.

In another aspect, the present invention encompasses methods of making one or more of the embodiments described herein, for example, a complex of an antibody that recognizes a tumor, and an enzyme that is able to create reactive oxygen species. In still another aspect, the present invention encompasses methods of using one or more of the embodiments described herein, for example, a complex of an antibody that recognizes a tumor, and an enzyme that is able to create reactive oxygen species.

Other advantages and novel features of the present invention will become apparent from the following detailed description of various non-limiting embodiments of the invention when considered in conjunction with the accompanying figures.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1B illustrate lysosomal function in normal cellular function and in malignant cells following uptake of oxidized LDL into the lysosome through endocytosis, in accordance with certain embodiments of the invention;

FIGS. 2A-2B illustrate cellular components within the autolysosome that are degraded and released into the cytoplasm, in contrast to autophagic-induced apoptosis, in another embodiment of the invention; and

FIG. 3 illustrates prophetic assays useful for determining immunogenic cell death, in yet another embodiment of the invention.

### DETAILED DESCRIPTION

The present invention generally relates to systems and methods for treating cancer using immunogenic cell death, e.g., lysosome-induced immunogenic cell death. This includes at least three aspects, which may be used separately or together. A first aspect involves the preparation of a tumor for treatment, for example, by withdrawal and suppression of antioxidants, supply of n-3 through n-6 and other unsaturated fatty acids, and/or treatment of the subject with statins. A second aspect involves restoration of p53 functionality through genetic manipulation, including techniques involving CRISPR. A third aspect involves constructing an antibody-enzyme complex where the antibody recognizes the tumor and the enzyme is an oxidase. The complex may be administered to a subject. The subject may also be provided with a substrate to the enzyme. These and/or other aspects, may be used separately or together, and may be used to treat and/or cure cancer. In some cases, the lysosome may be targeted in inducing cell death, e.g., in cancer cells.

As a non-limiting example, certain embodiments of the present invention are generally directed to antibodies that recognizes a tumor, which may be complexed to an enzyme, e.g., via a linkage, such as a covalent bond. The tumor may be for example, a tumor found in a subject having colorectal cancer. In some cases, colorectal cancers may be particularly difficult to treat, e.g., some treatments such as checkpoint inhibitors may be generally ineffective. Thus, compositions such as those described herein may be useful for treating such cancers. For example, in one embodiment, a composition as described herein may create antigens, e.g., by increase lysosomal membrane permeability and/or leakage, and/or by creating reactive oxygen species, etc., as discussed herein. This may allow the immune system of the subject, e.g., T cells, to recognize the tumor by recognizing the antigens created within the tumor of the subject. Accordingly, as a non-limiting example, in one embodiment, the composition comprises an anti-epCAM antibody, which may be able to target the tumor, e.g., the colorectal cancer. The composition also can comprise a suitable oxidase that is able to create antigens, e.g., as described herein. For instance, in one embodiment, the composition comprises xanthine oxidase. In some cases, the anti-epCAM antibody and the xanthine oxidase are covalently linked together, e.g., via a sulfhydryl-to-sulfhydryl bond. As another example, the linkage may be an amine-to-sulfhydryl bond. As yet another example, the linkage may be a hydroxyl-to-sulfhydryl bond. Other possible linkages include any of those described herein. In addition, in some cases, additional treatment steps may be used. For instance, in one embodiment, a checkpoint inhibitor, such as a PD-1 inhibitor, is used, e.g., in addition to the above composition.

It should be understood, however, that other compositions are also possible in addition to the above, e.g., to treat various cancers such as those described herein. For example, in certain embodiments, the present invention is generally directed to systems and methods for targeting the lysosomes, e.g., within tumors. Without wishing to be bound by any theory, it is believed that the lysosomes may be induced to become permeable and leak out degradative enzymes, which may produce antigens that can be recognized by the immune system. Thus, for example, in some cases, targeted lysosomes may cause the production of antigens that can be recognized by the immune system, which can allow the immune system to recognize the lysosome, and/or tumors containing the lysosomes. In such a fashion, a “cold” tumor that is not easily recognized by the immune system may be turned into a “hot” tumor more easily recognized by the immune system through the production of antigens. Thus, by creating antigens by causing leakage within lysosomes, a tumor can be treated, at least in certain embodiments of the invention.

It should, however, be understood that the above discussion is a non-limiting example of one embodiment of the present invention that can be used to treat a subject having cancer For example, some aspects are generally directed to preparing a tumor for treatment. For instance, pretreatment of the subject may prepare the subject for successful attack on the lysosome membrane, which may be useful for treating the tumor. Thus, the tumor may be prepared in some embodiments by supplying fatty acids, statins, etc. to the subject, and/or by removing or suppressing antioxidants from the subject. Without wishing to be bound by any theory, it is believed that, as discussed below, oxidization processes may be useful for treating the tumor. Accordingly, antioxidants may reduce or inhibit such tumor treatments, and thus, in some cases, the concentration of antioxidants within the patient may be lowered, e.g., to facilitate treatment. However, it should also be understood that pretreatment of a subject is not required in all embodiments, and in some cases, no pretreatment may be used.

A variety of methods may be used to prepare a tumor for treatment, for example, including withdrawal and suppression of antioxidants, supplying n-3 through n-6 and other unsaturated fatty acids, and/or treatment of the subject with statins. Without wishing to be bound by any theory, it is believed that pretreatment of the subject prepares for successful attack on the lysosome membrane. The antioxidant status of the subject may be lowered. Unsaturated fatty acids including n-3 and n-6 fatty acids are fed to the subject and/or the subject is given statins. These treatments may provide for more efficient attack on the lysosome membrane. However, it should be understood that the preparation of a tumor for treatment, e.g., as discussed herein, is not necessarily required in all embodiments.

In one set of embodiments, for example, unsaturated fatty acids are administered to the subject, e.g., fed to the subject. One or more fatty acids may be administered to a subject, e.g., simultaneously and/or sequentially. Typically, the unsaturated fatty acid may include one or more double bonds and/or triple bonds within the fatty acid chain. In some cases, the fatty acids may be relatively small-chain fatty acids. Non-limiting examples of such unsaturated fatty acids include C₃, C₄, C₅, C₆, C₇, and C₈ fatty acids. These may include monounsaturated and/or polyunsaturated fatty acids. Specific examples include, but are not limited to, CH₂═CHCOOH, CH₃CH═CHCOOH, CH₃CH₂CH═CHCOOH, CH₃CH₂CH₂CH═CHCOOH, CH₂═CHCH₂COOH, CH₂═CH CH₂CH₂COOH, CH₂═CHCH₂CH₂CH₂COOH, CH₂═CHCH₂CH═CHCOOH, CH₂═CHCH═CHCOOH, etc. In addition, in some cases, the fatty acids may come from naturally occurring sources, such as krill oil, fish oil, safflower oil, soybean oil, flaxseed oil, canola oil, algal oil, etc.

A wide range of dosing of fatty acids may be used. In some embodiments, fatty acids may be given to a subject, such as a human, at a dosage of at least 0.1 g, at least 0.2 g, at least 0.3 g, at least 0.4 g, at least 0.5 g, at least 0.6 g, at least 0.7 g, at least 0.8 g, at least 0.9 g, at least 1 g, at least 2 g, at least 3 g, at least 4 g, at least 5 g, at least 6 g, at least 7 g, at least 8 g, at least 9 g, at least 10 g, at least 11 g, at least 12 g, at least 13 g, at least 14 g, or at least 15 g. In some embodiments, the fatty acids may be given at a dosage of no more than 15 g, no more than 14 g, no more than 13 g, no more than 12 g, no more than 11 g, no more than 10 g, no more than 9 g, no more than 8 g, no more than 7 g, no more than 6 g, no more than 5 g, no more than 4 g, no more than 3 g, no more than 2 g, no more than 1 g, no more than 0.9 g, no more than 0.8 g, no more than 0.7 g, no more than 0.6 g, no more than 0.5 g, no more than 0.4 g, no more than 0.3 g, no more than 0.2 g, or no more than 0.1 g. Combinations of any of these are also possible in some embodiments, e.g., the dosage may be between 1 and 15 g, between 1 and 10 g, between 5 and 10 g, between 0.5 and 1 g, etc.

In some embodiments, the fatty acids may be given to a subject, such as a human, at a dosage of at least 1 mg/kg, at least 2 mg/kg, at least 3 mg/kg, at least 5 mg/kg, at least 10 mg/kg, at least 20 mg/kg, at least 30 mg/kg, at least 50 mg/kg, at least 100 mg/kg, at least 200 mg/kg, at least 300 mg/kg, at least 500 mg/kg, at least 1 g/kg, or at least 2 mg/kg. In some cases, the fatty acids may be given at a dosage of no more than 2 g/kg, no more than 1 g/kg, no more than 500 mg/kg, no more than 300 mg/kg, no more than 200 mg/kg, no more than 100 mg/kg, no more than 50 mg/kg, no more than 30 mg/kg, no more than 20 mg/kg, no more than 10 mg/kg, no more than 5 mg/kg, no more than 3 mg/kg, no more than 2 mg/kg, or no more than 1 mg/kg. Combinations of any of these are also possible in some embodiments, e.g., the dosage may be 1 to 2 g/kg, 500 mg/kg to 1 g/kg, 400 to 800 mg/kg, etc.

In another set of embodiments, the tumor may be prepared by administering statins to the subject, e.g., in addition to or instead of fatty acids. Statins, also known as HMG-CoA reductase inhibitors, are a class of lipid-lowering medications. Non-limiting examples of statins include atorvastatin, fluvastatin, lovastatin, pitavastatin, pravastatin, rosuvastatin, cerivastatin, mevastatin, and simvastatin. One or more statins may be administered to a subject, e.g., simultaneously and/or sequentially.

A wide range of dosing of statins may be used. For example, the statins may be given to a subject, such as a human, at a dosage of at least 1 mg, at least 2 mg, at least 3 mg, at least 5 mg, at least 10 mg, at least 20 mg, at least 30 mg, at least 40 mg, at least 50 mg, at least 60 mg, at least 70 mg, at least 80 mg, at least 90 mg, or at least 100 mg. In some cases, the statins may be given at a dosage of no more than 100 mg, no more than 90 mg, no more than 80 mg, no more than 70 mg, no more than 60 mg, no more than 50 mg, no more than 40 mg, no more than 30 mg, no more than 20 mg, no more than 10 mg, no more than 5 mg, no more than 3 mg, no more than 2 mg, or no more than 1 mg. Combinations of any of these are also possible in some embodiments, e.g., the dosage may be 10 to 20 mg, 20 to 40 mg, 40 to 80 mg, 5 to 10 mg, etc.

In addition, in some embodiments, statins may be given to a subject, such as a human, at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, or at least 15 mg/kg. In some embodiments, the statins may be given at a dosage of no more than 15 mg/kg, no more than 14 mg/kg, no more than 13 mg/kg, no more than 12 mg/kg, no more than 11 mg/kg, no more than 10 mg/kg, no more than 9 mg/kg, no more than 8 mg/kg, no more than 7 mg/kg, no more than 6 mg/kg, no more than 5 mg/kg, no more than 4 mg/kg, no more than 3 mg/kg, no more than 2 mg/kg, or no more than 1 mg/kg. Combinations of any of these are also possible in some embodiments, e.g., the dosage may be between 1 and 15 mg/kg, between 1 and 10 mg/kg, between 5 and 10 mg/kg, etc.

According to certain embodiments, if a subject is on antioxidants, the antioxidants may be removed or suppressed from the subject. For example, the antioxidants may be withheld entirely, or the antioxidant dosage may be reduced, e.g., in amount and/or frequency, as part of preparing the tumor for treatment. Non-limiting examples of antioxidants include beta carotene, vitamin A, and vitamin E. Other examples include vitamin C, glutathione, lipoic acid, uric acid, and ubiquinol.

It should be understood that in certain embodiments, any one or more of these may be independently used, separately or together. For example, to a subject may be administered statins, fatty acids, or both. If both, these may be administered separately and/or together. If separately, these may be administered simultaneously and/or sequentially, in any suitable order. Any suitable method of administration and dosing schedule may be used, including those discussed herein. Additionally, one or both may be present in a suitable pharmaceutically acceptable carrier. Furthermore, in some embodiments, antioxidants may be removed or suppressed from the subject, with or without the administration of statins, fatty acids, or both.

In some aspects, the systems and methods may be combined with checkpoint inhibitors, such as PD-1 inhibitors and/or PD-L1 inhibitors, to treat a tumor. For example, in some cases, systems and methods such as those described herein may cause leakage of lysosomes and/or the production of antigens, which can be recognized by the immune system. Without wishing to be bound by any theory, it is believed that the production of antigens, e.g., caused by lysosome leakage, may help the immune system to recognize the tumor, and thus more effectively attack it. In some cases, it is believed that such a reaction may be enhanced in the presence of checkpoint inhibitors, such as PD-1 inhibitors, thereby allowing the immune system to more effectively attack the tumor. Non-limiting examples of PD-1 or PD-L1 inhibitors include nivolumab, pembrolizumab, atezolizumab, avelumab, durvalumab, pidilizumab, cemiplimab, and the like. Other examples of checkpoint inhibitors that may be used include, but are not limited to, ipilimumab, ofatumumab, and rituximab. Various checkpoint inhibitors are commercially available. However, it should also be understood that in some embodiments, no checkpoint inhibitors are used.

The inhibitors, if present, may be provided in any suitable amount or concentration, and may be given simultaneously and/or sequentially with other compositions such as those described herein. Examples of dosages include, but are not limited to dosages of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, etc. In some embodiments, the dosage can be no more than 10 mg/kg, no more than 9 mg/kg, no more than 8 mg/kg, no more than 7 mg/kg, no more than 6 mg/kg, no more than 5 mg/kg, no more than 4 mg/kg, no more than 3 mg/kg, no more than 2 mg/kg, or no more than 1 mg/kg. Combinations of any of these are also possible in some embodiments, e.g., the dosage may be between 1 and 15 mg/kg, between 1 and 10 mg/kg, between 5 and 10 mg/kg, etc.

Other cancer immunotherapy treatments may also be used, e.g., in addition or instead of inhibitors such as checkpoint inhibitors. Non-limiting examples include non-specific immunotherapies, adaptive immunotherapies, monoclonal antibodies, CAR T-cell therapies, cancer vaccines, etc. In some cases, the cancer immunotherapy treatment may use and/or activate T cells, NK cells, and/or macrophages. These may be used, for example, in conjunction with the systems and methods described herein. Those of ordinary skill in the art will be familiar with systems and methods for producing monoclonal antibodies, identifying T cells, and the like.

For example, in CAR-T therapy, receptors combining antigen-binding and T-cell activating functions are prepared. The general premise of CAR-T cells is to artificially generate T-cells targeted to markers found on cancer cells. The T-cells are removed from the subject, genetically altered to target cancer cells, then returned to t subject. CAR-T cells create a link between an extracellular ligand recognition domain to an intracellular signaling molecule which in turn activates T cells. The extracellular ligand recognition domain may be a single-chain variable fragment (scFv). The CAR-T cells can be programmed to target antigens that are present on the surface of tumors. When they come in contact with the antigens on the tumors, the CAR-T cells are activated via binding to the tumor antigen. The CAR-T cells destroy the cancer cells through mechanisms such as extensive stimulated cell proliferation, increasing the degree to which the cell is toxic to other living cells i.e. cytotoxicity, and by causing the increased production of factors that are secreted from cells in the immune system that have an effect on other cells in the organism. These factors include cytokines, interleukins, interferons, growth factors, and the like.

Another aspect of the present invention involves restoration of p53 functionality through genetic manipulation, including techniques involving CRISPR. This may be performed independently or in conjunction with preparing a tumor for treatment, as discussed above, and if performed in conjunction, these may be performed simultaneously and/or sequentially, in any suitable order. Without wishing to be bound by any theory, it is believed that in many cancers, p53 is either non-functional or of diminished function. It may be important in the mechanism of attack of the lysosome membrane. Restoring its function by genetic methodology such as CRISPR may improve the ability of therapies to attack the lysosome membrane. However, as noted above, this is not necessarily required in all embodiments.

Yet another aspect of the present invention is directed to complexes such as antibody-enzyme complexes. Such complexes may be used in association with one or more of the above-described aspects, or independently of such aspects. The antibody may be one that recognizes the tumor. The enzyme may be an oxidase, or other enzyme that is able to create reactive oxygen species. The complex may be administered to a subject, for example, via infusion or other administration techniques such as those described herein. The subject may also be provided with a substrate to the enzyme in some cases. Without wishing to be bound by any theory, it is believed that the antibody helps to localize the complex within or near the tumor. Once localized, the substrate of the enzyme may be administered to the subject, e.g., infused, which may set off a series of events that result in attack of the lysosome membrane, release of lysosomal contents into the cell and cell death through apoptosis. Thus, certain embodiments of the invention are directed to lysosome-induced immunogenic cell death.

In addition, it should also be understood that besides enzymes, other therapeutic moieties may be used. For example, a complex of an antibody and a drug, or another targeting moiety and a drug, may be used. In one set of embodiments, the complex may be an antibody-drug conjugate (or ADC).

Those of ordinary skill in the art will be aware of antibodies, including those that are able to target tumors. For example, in one set of embodiments, the antibody may be an antibody to a tumor antigen. Examples of tumor-specific antigens include products of ras and p53, CTAG1B, or MAGEAL. Other examples include tissue differentiation antigens, mutant protein antigens, oncogenic viral antigens, cancer-testis antigens and vascular or stromal specific antigens. Other examples include oncofetal antigens, such as alphafetoprotein (AFP) and carcinoembryonic antigen (CEA). Still other examples include antibodies to CA-125, MUC-1, epithelial tumor antigen, tyrosinase, melanoma-associated antigen (MAGE), epithelial cell adhesion molecule (epCAM), etc.

Antibodies are typically proteins or glycoproteins of one or more polypeptides substantially encoded by immunoglobulin genes or fragments of immunoglobulin genes. The recognized immunoglobulin genes include the kappa, lambda, alpha, gamma, delta, epsilon, and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. Antibodies exist as intact immunoglobulins or as a number of well characterized fragments produced by digestion with various peptidases. Thus, for example, pepsin digests an antibody below (i.e. toward the Fc domain) the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region thereby converting the (Fab′)2 dimer into an Fab′ monomer. The Fab′ monomer is essentially a Fab with part of the hinge region. While various antibody fragments are defined in terms of the digestion of an intact antibody, such fragments may also be synthesized de novo, for example, chemically by utilizing recombinant DNA methodology, by “phage display” methods, or the like. Examples of antibodies include single chain antibodies, e.g., single chain Fv (scFv) antibodies in which a variable heavy and a variable light chain are joined together (directly or through a peptide linker) to form a continuous polypeptide. In one embodiment, the antibody is a monoclonal antibody.

The antibody and the enzyme may be linked or conjugated together, for example covalently. These may be directly bound to each other, or bound via one or more cross-linking agents. Non-limiting examples include glutaraldehyde, NHS-esters (N-hydroxysuccinimide) (e.g., dithiobis(succinimidylpropionate), dithiobis(sulfosuccinimidylpropionate), etc.), PEG groups, imidoesters (e.g., dimethyl adipimidate, dimethyl suberimidate, dimethyl pimelimidate, etc.), maleimides, pyridyls, carbodiimide, isocyanate, or the like. The antibody and the enzyme may be coupled through any suitable system, e.g., amine-to-amine, sulfhydryl-to-sulfhydryl, amine-to-sulfhydryl, carboxyl-to-amine, sulfhydryl-to-carbohydrate, hydroxyl-to-sulfhydryl, or the like. Those of ordinary skill in the art will be familiar with methods of cross-linking or conjugating proteins to each other.

As mentioned, the antibody may be linked to an enzyme. In one set of embodiments, the enzyme may be oxidase or a peroxidase. Oxidases are typically enzymes that catalyze an oxidation-reduction reaction, including those involving oxygen (O₂) as the electron acceptor. The oxygen may be reduced to water (H₂O), hydrogen peroxide (H₂O₂). Non-limiting examples include glucose oxidase, monoamine oxidase, cytochrome P450 oxidase, NADPH oxidase, xanthine oxidase, L-gulonolactone oxidase, laccase, lysyl oxidase, or the like. Similarly, peroxidases are enzymes that may act on substrates such as hydrogen peroxide or lipid peroxides. Non-limiting examples include horseradish peroxidase, cytochrome c peroxidase, ascorbate peroxidase, chloride peroxidase, glutathione peroxidase, haloperoxidase, lactorperoxidase, manganese peroxidase, myeloperoxidase, thyroid peroxidase, vanadium boromoperoxidase, or the like.

In some cases, a substrate for the enzyme may also be administered to the subject. The substrate and the enzyme may be administered in any suitable order, e.g., sequentially and/or simultaneously. Non-limiting examples of substrates include hypoxanthine or xanthine for xanthine oxidase, or glucose for glucose oxidase. As additional examples, monoamine oxidase may act on serotonin, melatonin, norepinephrine, epinerphrine, phenethylamine, benzylamine, dopamine, tyramine, tryptamine, etc. for monoamine oxidase; NADPH for NADPH peroxidase; ascorbate for ascorbate peroxidase; or the like.

Without wishing to be bound by any theory, it is believed that the oxidase or peroxidase acts by creating reactive oxygen species such as superoxides, peroxides, hydroxyl radicals, etc. by oxidizing its substrate. Accordingly, in some embodiments, a substrate may be optionally added to facilitate such reactions. The reactive oxygen species may interact with the lysosome of the tumor cells, inducing the lysosomal membrane to become more permeable and leak out degradative enzymes, which may cause digestion or other reactions within the cell. For example, reactive oxygen species such as superoxides may cause oxidation of cellular components, e.g., unsaturated lipids, which may be endocytosed and destabilize lysosome membranes, dispersing lysosomal enzymes throughout the cell, which then creates tumor-specific neoantigens that can be recognized by the immune system, and/or which may trigger apoptosis or cell death. Thus, such reactive oxygen species may be useful in some embodiments to cause lysosome-induced immunogenic cell death. Accordingly, it is believed that tumor cells may be targeted (e.g., with an antibody) for the delivery of enzymes able to create reactive oxygen species, which may then cause leaking of lysosomes and ultimately the death of the tumor cells. See also Example 1, below.

In some cases, such enzymes may be facilitated by preparing the tumor for treatment, e.g., using fatty acids and/or statins, and/or by removing or suppressing antioxidants from the subject. In addition, in some cases, p53 functionality may be restored through genetic manipulation.

In addition, it should be understood that in some cases, a drug may be used within the complex, instead and/or in addition to an enzyme that creates a reactive oxygen species.

The complexes may be administered to the subject, such as a human, using any suitable technique, including those described herein. In addition, a wide range of doses may be used. For example, the complex may be applied at a dosage of at least 10 mg, at least 15 mg, at least 20 mg, at least 25 mg, at least 30 mg, at least 40 mg, at least 50 mg, at least 60 mg, at least 70 mg, at least 80 mg, at least 90 mg, at least 100 mg, at least 200 mg, at least 300 mg, at least 500 mg, at least 1000 mg, at least 1500 mg, at least 2000 mg, at least 2500 mg, at least 3000 mg, at least 5000 mg, etc. In some embodiments, the complex may be applied at a dosage of no more than 5000 mg, no more than 3000 mg, no more than 2500 mg, no more than 2000 mg, no more than 1500 mg, no more than 1000 mg, no more than 500 mg, no more than 300 mg, no more than 200 mg, no more than 100 mg, no more than 90 mg, no more than 80 mg, no more than 70 mg, no more than 60 mg, no more than 50 mg, no more than 40 mg, no more than 30 mg, no more than 25 mg, no more than 20 mg, no more than 15 mg, no more than 10 mg, etc. Combinations of any of these are also possible in some embodiments, e.g., the dosage may be 10 to 20 mg, 50 to 100 mg, 100 to 200 mg, or the like.

In addition, in some embodiments, the complexes may be given to a subject, such as a human, at a dosage of at least 1 mg/kg, at least 2 mg/kg, at least 3 mg/kg, at least 4 mg/kg, at least 5 mg/kg, at least 6 mg/kg, at least 7 mg/kg, at least 8 mg/kg, at least 9 mg/kg, at least 10 mg/kg, at least 11 mg/kg, at least 12 mg/kg, at least 13 mg/kg, at least 14 mg/kg, or at least 15 mg/kg. In some embodiments, the statins may be given at a dosage of no more than 15 mg/kg, no more than 14 mg/kg, no more than 13 mg/kg, no more than 12 mg/kg, no more than 11 mg/kg, no more than 10 mg/kg, no more than 9 mg/kg, no more than 8 mg/kg, no more than 7 mg/kg, no more than 6 mg/kg, no more than 5 mg/kg, no more than 4 mg/kg, no more than 3 mg/kg, no more than 2 mg/kg, or no more than 1 mg/kg. Combinations of any of these are also possible in some embodiments, e.g., the dosage may be between 1 and 15 mg/kg, between 1 and 10 mg/kg, between 5 and 10 mg/kg, etc.

In another aspect, a composition as described herein may be administered to a subject, either by itself and/or in conjunction with co-factors, other therapeutics, or the like. For example, an antibody-enzyme complex (e.g., comprising an oxidase, or other enzyme able to create a reactive oxygen species) may be administered alone, or in conjunction with substrates such as hypoxanthine, xanthine, glucose, or the like, e.g., as discussed herein, and/or in conjunction with fatty acids, statins, etc. When administered, the compositions can be applied in a therapeutically effective, pharmaceutically acceptable amount as a pharmaceutically acceptable formulation, for example, a pharmaceutically acceptable carrier such as those described below. The term “effective amount” of a composition, such as the complexes described herein, refers to the amount necessary or sufficient to realize a desired biologic effect. For example, an effective amount of a complex to treat a tumor may be an amount sufficient to reduce the tumor's size. Combined with the teachings provided herein, by choosing among the various active compositions and weighing factors such as potency, relative bioavailability, patient body weight, severity of adverse side effects and mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular compositions being administered the size of the subject, or the severity of the disease or condition. One of ordinary skill in the art can empirically determine the effective amount of the compositions without necessitating undue experimentation.

The terms “treat,” “treated,” “treating,” and the like, when used herein, refer to administration of the compositions to a subject which may increase the resistance of the subject to development or further development of the tumor, to administration of the composition after the subject has developed the tumor in order to eliminate or at least control development of the tumor, and/or slow the progression of or to reduce the severity of symptoms caused by the tumor. When administered to a subject, effective amounts may depend on the particular tumor being treated and the desired outcome. A therapeutically effective dose may be determined by those of ordinary skill in the art, for instance, employing factors such as those further described below and using no more than routine experimentation.

For use in therapy, an effective amount of the compositions can be administered to a subject by any mode that delivers the composition to the tumor, e.g., mucosal, systemic, or the like. Administering the pharmaceutical composition of the present invention may be accomplished by any means known to the skilled artisan. Example routes of administration include but are not limited to oral, parenteral, intramuscular, intranasal, sublingual, intratracheal, inhalation, ocular, vaginal, intravenously, percutaneously, and rectal. In some cases, intramuscular administration is used. In one set of embodiments, intravenous administration is used.

In administering the compositions to a subject, dosing amounts, dosing schedules, routes of administration, and the like may be selected so as to affect known activities of these compositions. Dosages may be estimated based on the results of experimental models, optionally in combination with the results of assays of compositions described herein. Dosage may be adjusted appropriately to achieve desired drug levels, local or systemic, depending upon the mode of administration. The doses may be given in one or several administrations per day. Multiple doses per day are also contemplated in some cases to achieve appropriate systemic levels of the compositions within the subject or within the tumor.

The dose of the compositions to the subject may be such that a therapeutically effective amount of the compositions reaches the tumor within the subject. The dosage may be given in some cases at the maximum amount while avoiding or minimizing any potentially detrimental side effects within the subject. The dosage of the compositions actually administered may be dependent upon factors such as the final concentration desired at the tumor, the method of administration to the subject, the efficacy of the composition, the longevity of the composition within the subject, the timing of administration, the effect of concurrent treatments (e.g., as in a cocktail), etc. The dose delivered may also depend on conditions associated with the subject, and can vary from subject to subject in some cases. For example, the age, sex, weight, size, environment, physical conditions, or current state of health of the subject may also influence the dose required and/or the concentration of the composition at the active site. Variations in dosing may occur between different individuals or even within the same individual on different days. In some cases, a maximum dose be used, that is, the highest safe dose according to sound medical judgment. In some cases, the dosage form is such that it does not substantially deleteriously affect the subject.

Subject doses of the compounds described herein for delivery may range from about 0.1 microgram to 10 mg per administration, which depending on the application could be given daily, weekly, or monthly and any other amount of time therebetween. In some cases, doses range from about 10 microgram to 5 mg per administration, e.g., from about 100 microgram to 1 mg, with 2 to 4 administrations being spaced days or weeks apart. In some embodiments, doses range from 1 microgram to 10 mg per administration, and most typically 10 microgram to 1 mg, with daily or weekly administrations. Other suitable dosings have been described in detail herein.

The compositions may be administered in multiple doses over extended period of time. For any compound described herein the therapeutically effective amount can be initially determined from animal models. The applied dose can be adjusted based on the relative bioavailability and potency of the administered compound. Adjusting the dose to achieve maximal efficacy based on the methods described above and other methods as are well-known in the art is well within the capabilities of the ordinarily skilled artisan.

The treatments disclosed herein may be given to any subject, for example, a human, or a non-human animal, such as a dog, a cat, a horse, a rabbit, a cow, a pig, a sheep, a goat, a rat (e.g., Rattus Norvegicus), a mouse (e.g., Mus musculus), a guinea pig, a non-human primate (e.g., a monkey, a chimpanzee, a baboon, an ape, a gorilla, etc.), or the like.

In certain embodiments, the compositions can be administered to a subject who has a family history of cancer, or to a subject who has a genetic predisposition for cancer. In other embodiments, the compositions can be administered to a subject who has reached a particular age, or to a subject more likely to get a tumor. In yet other embodiments, the compositions are administered to subjects who exhibit symptoms of cancer (e.g., early or advanced). In still other embodiments, the compositions may be administered to a subject as a preventive measure. In some embodiments, the compositions may be administered to a subject based on demographics or epidemiological studies, or to a subject in a particular field or career. Non-limiting examples of cancers that may be treated include brain, pancreatic, blood, melanoma, multiple melanoma, lung, breast, prostate, ovarian, colon, colorectal, glioblastoma, pancreas, and other cancers.

Administration of a composition of the invention may be accomplished by any medically acceptable method which allows the composition to reach its target, e.g., a tumor. The particular mode selected may depend of course, upon factors such as those previously described, for example, the particular composition, the severity of the state of the subject being treated, the dosage required for therapeutic efficacy, etc. As used herein, a “medically acceptable” mode of treatment is a mode able to produce effective levels of the compositions within the subject without causing clinically unacceptable adverse effects.

Any medically acceptable method may be used to administer the compositions to the subject. The administration may be localized (i.e., to a particular region, physiological system, tissue, organ, or cell type) or systemic, depending on the condition to be treated. For example, the compositions may be administered orally, vaginally, rectally, buccally, pulmonary, topically, nasally, transdermally, through parenteral injection or implantation, via surgical administration, or any other method of administration where access to the tumor is achieved. In some cases, more than one method of administration may be used, e.g., if two or more compositions are to be administered.

Examples of parenteral modalities that can be used include intravenous, intradermal, subcutaneous, intracavity, intramuscular, intraperitoneal, epidural, or intrathecal. Examples of implantation modalities include any implantable or injectable drug delivery system. Oral administration may be used in some embodiments because of the convenience to the subject as well as the dosing schedule. Compositions suitable for oral administration may be presented as discrete units such as hard or soft capsules, pills, cachettes, tablets, troches, or lozenge. Other oral compositions suitable for use include solutions or suspensions in aqueous or non-aqueous liquids such as a syrup, an elixir, or an emulsion. In another set of embodiments, a composition may be used to fortify a food or a beverage.

The compositions, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions, or emulsions in oily or aqueous vehicles, and may contain formulatory agents such as suspending, stabilizing and/or dispersing agents.

Pharmaceutical formulations for parenteral administration include aqueous solutions of the active compounds in water soluble form. Additionally, suspensions of the active compounds may be prepared in some cases as appropriate oily injection suspensions. Suitable lipophilic solvents or vehicles include, but are not limited to, fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

In addition to the formulations described previously, the compositions may also be formulated as a depot preparation in some embodiments. Such long-acting formulations may be formulated in some cases with suitable polymeric or hydrophobic materials (for example, as an emulsion in an acceptable oil) or ion exchange resins, or as sparingly soluble derivatives, for example, as a sparingly soluble salt.

Suitable liquid pharmaceutical preparation forms include, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin. The pharmaceutical compositions also may include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrants, binders, coating agents, swelling agents, lubricants, flavorings, sweeteners or solubilizers are customarily used as described above.

In certain embodiments of the invention, the administration of a composition as described herein may be designed so as to result in sequential exposures to the composition over a certain time period, for example, hours, days, weeks, months or years. This may be accomplished, for example, by repeated administrations of the composition by one of the methods described herein, or by a sustained or controlled release delivery system in which a composition is delivered over a prolonged period without repeated administrations. Administration of a composition using such a delivery system may be, for example, by oral dosage forms, bolus injections, transdermal patches, subcutaneous implants, or other methods such as those described herein. Maintaining a substantially constant concentration of a composition may be desired in some cases.

Other delivery systems suitable for use in certain embodiments include time-release, delayed release, sustained release, or controlled release delivery systems. Such systems may avoid repeated administrations in many cases, increasing convenience to the subject. Many types of release delivery systems are available and known to those of ordinary skill in the art. They include, for example, polymer-based systems such as polylactic and/or polyglycolic acids, polyanhydrides, polycaprolactones and/or combinations of these; nonpolymer systems that are lipid-based including sterols such as cholesterol, cholesterol esters, and fatty acids or neutral fats such as mono-, di- and triglycerides; hydrogel release systems; liposome-based systems; phospholipid based-systems; silastic systems; peptide based systems; wax coatings; compressed tablets using conventional binders and excipients; or partially fused implants. Specific examples include, but are not limited to, erosional systems in which the composition is contained in a form within a matrix, or diffusional systems in which an active component controls the release rate. The formulation may be present as, for example, microspheres, hydrogels, polymeric reservoirs, cholesterol matrices, or polymeric systems, etc. In some embodiments, the system may allow sustained or controlled release of a composition to occur, for example, through control of the diffusion or erosion/degradation rate of the formulation. In addition, a pump-based hardware delivery system may be used in some embodiments.

Use of a long-term release implant may be particularly suitable in some embodiments. “Long-term release,” as used herein, means that an implant containing a composition as described herein is constructed and arranged to deliver therapeutically effective levels for at least 30 or 45 days, or at least 60 or 90 days, or even longer in some cases. Long-term release implants are well known to those of ordinary skill in the art, and include some of the release systems described herein.

Administration of the compositions as described herein can be alone, or in combination with other therapeutic agents and/or compositions. For example, non-limiting examples of anti-cancer agents and drugs that can be used in combination with various compositions described herein include, but are not limited to, any one or more of 20-epi-1,25 dihydroxyvitamin D3, 4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, cam 700, cartilage derived inhibitor, carubicin hydrochloride, carzelesin, casein kinase inhibitors, castanospermine, cecropin B, cedefingol, cetrorelix, chlorambucil, chlorins, chloroquinoxaline sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatin, dacarbazine, dacliximab, dactinomycin, daunorubicin hydrochloride, decitabine, dehydrodidemnin B, deslorelin, dexifosfamide, dexormaplatin, dexrazoxane, dexverapamil, dezaguanine, dezaguanine mesylate, diaziquone, didemnin B, didox, diethylnorspermine, dihydro-5-azacytidine, dioxamycin, diphenyl spiromustine, docetaxel, docosanol, dolasetron, doxifluridine, doxorubicin, doxorubicin hydrochloride, droloxifene, droloxifene citrate, dromostanolone propionate, dronabinol, duazomycin, duocarmycin SA, ebselen, ecomustine, edatrexate, edelfosine, edrecolomab, eflornithine, eflornithine hydrochloride, elemene, elsamitrucin, emitefur, enloplatin, enpromate, epipropidine, epirubicin, epirubicin hydrochloride, epristeride, erbulozole, erythrocyte gene therapy vector system, esorubicin hydrochloride, estramustine, estramustine analog, estramustine phosphate sodium, estrogen agonists, estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabine phosphate, fluorodaunorunicin hydrochloride, fluorouracil, flurocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idarubicin hydrochloride, idoxifene, idramantone, ifosfamide, ilmofosine, ilomastat, imidazoacridones, imiquimod, immunostimulant peptides, insulin-like growth factor-1 receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomalcin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/myobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazinomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RII retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI 1 mimetics, semustine, senescence derived inhibitor 1, sense oligonucleotides, signal transduction inhibitors, signal transduction modulators, simtrazene, single chain antigen binding protein, sizofiran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin inhibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, and zorubicin hydrochloride, as well as salts, homologs, analogs, polymorphs, derivatives, enantiomers, and/or functionally equivalent compositions thereof.

In certain embodiments of the invention, a composition can be combined with a suitable pharmaceutically acceptable carrier, for example, as incorporated into a liposome, incorporated into a polymer release system, or suspended in a liquid, e.g., in a dissolved form or a colloidal form, or other methods such as those described herein. In general, pharmaceutically acceptable carriers suitable for use are well-known to those of ordinary skill in the art. As used herein, a “pharmaceutically acceptable carrier” refers to a non-toxic material that does not significantly interfere with the effectiveness of the biological activity of the active compound(s) to be administered, but is used as a formulation ingredient, for example, to stabilize or protect the active compound(s) within a composition before use. The carrier may include one or more compatible solid or liquid fillers, diluents or encapsulating substances which are suitable for administration to a human or other vertebrate animal. The term “carrier” denotes an organic or inorganic ingredient, which may be natural or synthetic, with which one or more active compounds of the invention are combined to facilitate application. The carrier may be co-mingled or otherwise mixed with one or more compositions as described herein, and/or with each other, in a manner such that there is no interaction which would substantially impair the desired pharmaceutical efficacy. The carrier may be either soluble or insoluble, depending on the application. Examples of well-known carriers include, but are not limited to, glass, polystyrene, polypropylene, polyethylene, dextran, nylon, amylase, natural and modified cellulose, polyacrylamide, agarose and magnetite. The nature of the carrier can be either soluble or insoluble.

The formulations described herein may be administered in pharmaceutically acceptable solutions in some embodiments, which may routinely contain pharmaceutically acceptable concentrations of salt, buffering agents, preservatives, compatible carriers, adjuvants, emulsifiers, diluents, excipients, chelating agents, fillers, drying agents, antioxidants, antimicrobials, preservatives, binding agents, bulking agents, silicas, solubilizers, stabilizers and optionally other therapeutic ingredients, that may be used with the active compound. For example, if the formulation is a liquid, the carrier may be a solvent, partial solvent, or non-solvent, and may be aqueous or organically based. Non-limiting examples of suitable formulation ingredients include diluents such as calcium carbonate, sodium carbonate, lactose, kaolin, calcium phosphate, or sodium phosphate; granulating and disintegrating agents such as corn starch or algenic acid; binding agents such as starch, gelatin or acacia; lubricating agents such as magnesium stearate, stearic acid, or talc; time-delay materials such as glycerol monostearate or glycerol distearate; suspending agents such as sodium carboxymethylcellulose, methylcellulose, hydroxypropylmethylcellulose, sodium alginate, polyvinylpyrrolidone; dispersing or wetting agents such as lecithin or other naturally-occurring phosphatides; thickening agents such as cetyl alcohol or beeswax; buffering agents such as acetic acid and salts thereof, citric acid and salts thereof, boric acid and salts thereof, or phosphoric acid and salts thereof; or preservatives such as benzalkonium chloride, chlorobutanol, parabens, or thimerosal. The compositions of the invention may be formulated into preparations in solid, semi-solid, liquid or gaseous forms such as tablets, capsules, elixirs, powders, granules, ointments, solutions, depositories, inhalants or injectables, etc.

Suitable buffering agents include, but are not limited to: acetic acid and a salt (1-2% w/v); citric acid and a salt (1-3% w/v); boric acid and a salt (0.5-2.5% w/v); and phosphoric acid and a salt (0.8-2% w/v). Suitable preservatives include, but are not limited to, benzalkonium chloride (0.003-0.03% w/v); chlorobutanol (0.3-0.9% w/v); parabens (0.01-0.25% w/v) and thimerosal (0.004-0.02% w/v).

Preparations include sterile aqueous or nonaqueous solutions, suspensions and emulsions, which can be isotonic with the blood of the subject in certain embodiments. Non-limiting examples of nonaqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include, but are not limited to, water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present in some embodiments, such as, for example, antimicrobials, antioxidants, chelating agents and inert gases and the like.

In some embodiments, a composition as described herein may be brought into association or contact with a suitable carrier, which may constitute one or more accessory ingredients. The final compositions may be prepared by any suitable technique, for example, by uniformly and intimately bringing a composition into association with a liquid carrier, a finely divided solid carrier or both, optionally with one or more formulation ingredients as previously described, and then, if necessary, shaping the product.

The compositions as discussed herein, and optionally other therapeutics, may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. The term “pharmaceutically acceptable salts” includes salts of compositions described herein, prepared in combination with, for example, acids or bases. Pharmaceutically acceptable salts can be prepared as alkaline metal salts, such as lithium, sodium, or potassium salts; or as alkaline earth salts, such as beryllium, magnesium or calcium salts. Examples of suitable bases that may be used to form salts include ammonium, or mineral bases such as sodium hydroxide, lithium hydroxide, potassium hydroxide, calcium hydroxide, magnesium hydroxide, and the like. Examples of suitable acids that may be used to form salts include inorganic or mineral acids such as hydrochloric, hydrobromic, hydroiodic, hydrofluoric, nitric, carbonic, monohydrogencarbonic, phosphoric, monohydrogenphosphoric, dihydrogenphosphoric, sulfuric, monohydrogensulfuric, phosphorous acids and the like. Other suitable acids include organic acids, for example, acetic, propionic, isobutyric, maleic, malonic, benzoic, succinic, suberic, fumaric, mandelic, phthalic, benzenesulfonic, p-tolylsulfonic, citric, tartaric, methanesulfonic, glucuronic, galacturonic, salicylic, formic, naphthalene-2-sulfonic, and the like. Still other suitable acids include amino acids such as arginate, aspartate, glutamate, and the like. Also, such salts can be prepared as alkaline metal or alkaline earth salts, such as sodium, potassium or calcium salts of the carboxylic acid group.

In another aspect, the present invention also provides any of the above-mentioned compositions in kits, optionally including instructions for use of the composition for the treatment of a tumor. In some cases, the kit can include a description of use of the compositions as discussed herein. The kit also can include instructions for use of a combination of two or more compositions. Instructions also may be provided for administering the compositions by any suitable technique as previously described, for example, orally, intravenously, pump or implantable delivery device, or via another known route of drug delivery.

The kits described herein may also contain one or more containers, which may contain compositions and other ingredients as previously described. The kits also may contain instructions for mixing, diluting, and/or administrating the compositions of the invention in some cases. The kits also can include other containers with one or more solvents, surfactants, preservative and/or diluents (e.g., normal saline (0.9% NaC), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided in some cases. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

In still another aspect, the invention includes the promotion of one or more of the above-described embodiments, e.g., in vitro or in vivo, promotion of treatment or prevention of a tumor, e.g., by administering, to a subject, compositions such as those described herein. As used herein, “promoted” includes all methods of doing business, including methods of education, scientific inquiry, academic research, industry activity including pharmaceutical industry activity, and any advertising or other promotional activity including written, oral and electronic communication of any form.

U.S. Provisional Patent Application Ser. No. 62/587,207, filed Nov. 16, 2017, entitled “Lysosome initiated Apoptiosis—A method for cancer therapy,” by Fossel, and U.S. Provisional Patent Application Ser. No. 62/740,477, filed Oct. 3, 2018, entitled “Systems and Methods for Initiating Apoptosis to Treat Cancer,” by Fossel, are each incorporated herein by reference in their entireties.

The following examples are intended to illustrate certain embodiments of the present invention, but do not exemplify the full scope of the invention.

### Example 1

One step in the progression to Immunogenic Cell Death (ICD) is the generation of neoantigens. Lysosomes contain various degradative enzymes including proteases, glycosidases and phosphatases. If released into the cytoplasm, these enzymes would degrade intracellular contents and in the process potentially would generate neoantigens. These would then be taken up by dendritic cells and macrophages. These cells travel to lymph nodes where the neoantigens activate T and B cells which then work to cause cell death by activation of the adaptive immune system.

### Example 2

In this prophetic example, to a human subject having a tumor, a composition is provided that comprises a complex comprising an antibody that recognizes a tumor, and an enzyme. In some cases, the antibody is one that can be obtained commercially, e.g., a tumor-recognizing antibody or a monoclonal antibody.

The enzyme is one that causes leakage of a lysosome, which may be lead to the production of antigens. In this example, the enzyme is an oxidase that produces reactive oxygen species, which damages or disrupts the lysosome and causes it to leak. The oxidase may be able to create a reactive oxygen species, such as a superoxide. In this example, the oxidase is xanthine oxidase.

The xanthine oxidase is complexed to the antibody via a covalent bond, and is administered to the subject intravenously or intramuscular. Upon targeting by the antibody to the tumor, the enzyme reacts with the lysosome, causing damage or disruption, allowing antigens to be created, which boost the immune response of the subject. In such fashion, the immune system of the subject is able to more effectively target the tumor of the subject.

### Example 3

In this prophetic example, a human subject is treated as in Example 2, but in addition, the subject is also exposed to a regimen to withdraw and/or suppress antioxidants, which could interfere with action of the complex. In one example, the subject is provided with an unsaturated fatty acid, such as CH₂═CHCOOH. The fatty acids may act to inhibit action of the antioxidants, leading to a larger response to the complex.

### Example 4

In this prophetic example, a human subject is treated as in Example 2, but in addition, the subject is exposed to a statin, such as atorvastatin, which may act to inhibit action of the antioxidants, leading to a larger response to the complex.

### Example 5

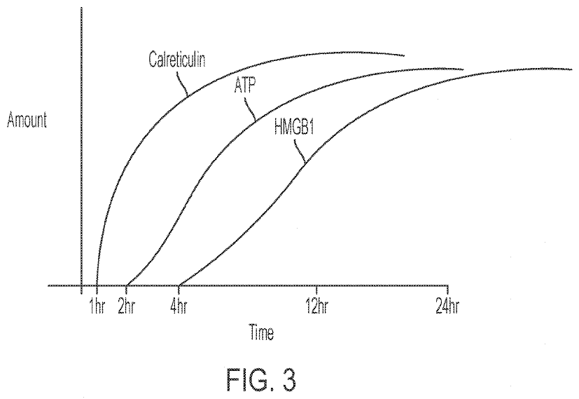

In this prophetic example, a conjugate between an epCAM recognizing antibody and the enzyme xanthine oxidase is prepared using an amino-to-amino crosslinking agent. MC26 malignant cells, which are recognized by anti-epCAM, are cultured in 96 well plates and treated with the conjugate at appropriate concentrations from 10 microgram/ml to 100 microgram/ml. Hypoxanthine was provided to the cell culture media at 1 mM concentration. There are recognized hallmarks of ICD. These include Calreticulin exposure, extra-cellular ATP formation, and HMGB1 formation. These are assayed for at 1 h, 2 h, 4 h, 6 h, 12 h and 24 h. A graph of the results is shown in FIG. 3. Appropriately calreticulin forms early followed by ATP formation and then followed by HMGB1 formation. This establishes that the cells produce the markers of ICD and are expected in the course of dying to form neoantigens which activate T cells and direct them to the tumor.

### Example 6

Once tumors are killed in a process which induces ICD the animal bearing the tumor is “immunized” to that tumor. Thus ICD protects the animal from that tumor in the future.

To demonstrate this, in this example, the tumor cells fragments killed in Example 2 were collected and injected into the left flank of BALB/c mice. Eight days after injection, live MC26 cells were injected into the right flank of each mouse. Mice were monitored for tumor growth in the right flank for 120 days. Mice treated with ICD dead cells were compared with mice that were injected with saline. At 120 days, 92% of the ICD dead cells were tumor free while only 7% of saline injected mice were tumor free.

### Example 7

It has been shown that Reactive Oxygen Species (ROS) can initiate a process that leads to lysosomal membrane permeabilization (LMP) causing release of lysosomal enzymes and cell death by apoptosis. The source of ROS, in particular superoxide, can be extracellular including in the tumor microenvironment and/or intracellular. The result of both are the same with respect to the lysosome and the resultant apoptosis, but the mechanisms are somewhat different. This example discusses these two mechanisms and their common outcome, Primary Cell Death (PCD) producing the neoantigens which are required for ICD.

There have been placebo-controlled studies which have shown that use of anti-oxidants in at-risk populations, rather than protecting subjects from cancer as expected, raised cancer incidence significantly. This is an important clue in implicating ROS in cancer cell death.

Malignant cells develop spontaneously and left unchecked progress to clinically detectable tumors. Early in tumor development, there is a balance between malignant cell growth and the ability of immune surveillance to kill these cells. The addition of antioxidants may suppress one line of immune defense and tipped that balance in favor of malignant cell growth. Antioxidants such as Vitamin E, beta carotene and Vitamin A interact with free radicals such as Reactive Oxygen Species (ROS) and inactivate them. This suggests that ROS such as superoxides produced by immune cells may play an important role in that natural defense against tumor development.

ROS such as superoxide react with various species, but importantly it easily oxidizes polyunsaturated fatty acids including those contained in LDL resulting in oxidized LDL (oxLDL). OxLDL is found in plasma of subjects at substantially higher levels than controls in subjects with breast, prostate, ovarian, colon and other cancers as a result of oxidation by free radicals such as superoxide. This oxLDL may result from the immune system attempting to fight the tumor. Suppressing the oxidation of lipids including the lipids of LDL may be the primary reason treatment with anti-oxidants increased the incidence of cancer.

It has been recently observed that the vast majority of all agents used to directly kill cancer cells (ionizing radiation, most chemotherapeutic agents and some targeted therapies) work either directly or indirectly by generating reactive oxygen species. Yet the path from initiation of therapy with these agents to cell death, often by apoptosis, are shrouded by substantial opacity. The lysosome, which has been lurking as a player in the treatment of cancer ever since its discovery, may play a role in this pathway.

Endocytosis mediated apoptosis. Oxidized LDL (oxLDL) circulates in the plasma of subjects with a variety of types of cancer including breast, ovarian, prostate and colorectal cancers, as well as other cancers such as those described herein. This oxLDL may be the result of reaction of extracellular ROS with circulating LDL. OxLDL may be what prevents antioxidants from forming. Oxidation of LDL in plasma may be associated with the host response of the body defending against the malignancy.

OxLDL is cytotoxic to malignant cells, and this cytotoxicity may be preferential to malignant cells compared to their non-malignant counterparts. It has been shown that oxLDL is taken up by endocytosis involving the LDL receptor. Malignant cells have been shown to have greater LDL receptor activity than their benign counterparts, perhaps because of their greater lipid requirements. Further, it has been shown that statins increase cellular uptake of LDL and in some cases increased activity of LDL receptors in the cell membrane, resulting in increased cytotoxicity of oxLDL in the malignant cells. There are clinical reports of improved efficacy of various cancer therapies with statin treatment.

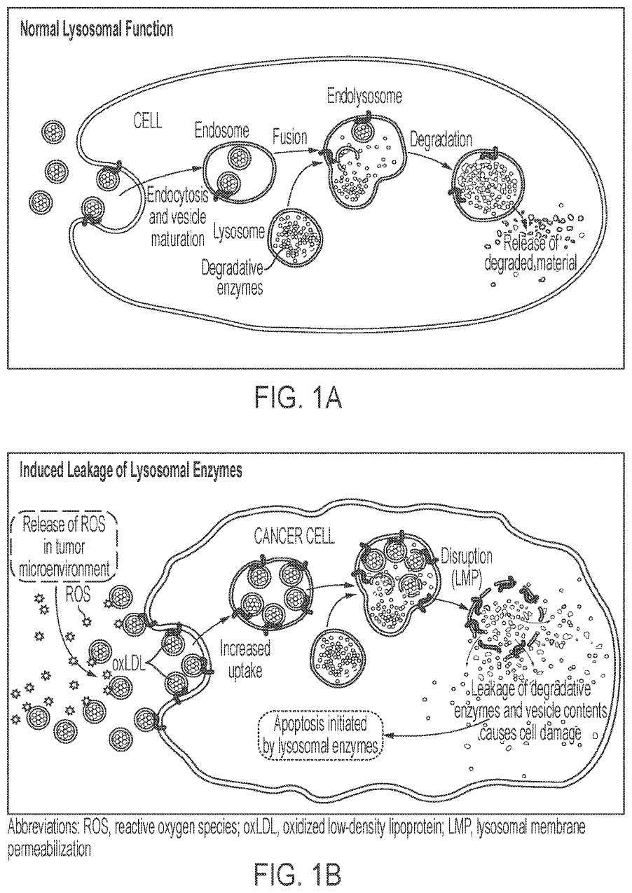

Using fluorescent labeling, it has been demonstrated that oxLDL was localized in lysosomes and that following lysosomal localization the lysosomal membrane became permeable and that released lysosomal contents digested cellular contents resulting in apoptosis. This is represented in the illustrations of FIG. 1 contrasting lysosomal function in normal cellular function (FIG. 1A) and in malignant cells following uptake of oxLDL into the lysosome through endocytosis (FIG. 1B). In FIG. 1A materials including LDL are taken into endosomes which then fuse with lysosomes and the endocytosed content is degraded within the lysosome and excreted into the cytoplasm as discarded fragments. Here, the lysosomal membrane remains intact.

In malignant cells, it has been demonstrated that oxLDL was taken up by the LDL receptor and incorporated into lysosomes. The incorporation of oxLDL and its attendant lipid peroxidation products may compromise the integrity of the lysosomal membrane and within a short time period the contents of the lysosome leaked into the cell in a process known as lysosomal membrane permeabilization (LMP). These leaked contents include degradative enzymes which, once free in the cytoplasm, digest the cell resulting in cell death through lysosome mediated apoptosis. In this process, new neoantigens of various composition would be formed. This process is illustrated in FIG. 1B. Electron micrographs of these cells exhibit the classic features of apoptosis resulting from other pathways.

FIG. 1A shows normal lysosome function degrades materials such as LDL by endocytosis, degrades the materials and excretes the degradants into the cytoplasm. It has been shown that oxidized LDL (oxLDL) taken up by endocytosis through the LDL receptor localized in lysosomes (FIG. 1B). The lysosome membrane becomes leaky as shown by pre-labeling of lysosomes with Lucifer Yellow and following the fluorescence over time. The contents of lysosomes including degradative enzymes are released into the cytoplasm degrading cellular contents and inducing apoptosis as shown by electron microscopy.

Autophagy mediated apoptosis. Many chemotherapeutic agents including anthracyclines initiate apoptotic cell death. It is believed that p53 results in apoptosis through a three-step process: (1) the transcriptional induction of redox-related genes; (2) the formation of reactive oxygen species; and (3) the oxidative degradation of mitochondrial components, culminating in cell death. The oxidative degradation may not be limited to mitochondrial components. Various chemotherapeutic agents with diverse modes of action including cisplatin, doxyrubicin, adriamycin, 5-fluorouricil, vincristine, and others may require functional p53 for activity. p53 senses damage to cellular components including DNA and activates the series of events described above leading to production of ROS and apoptosis. Often when efficacy of such chemotherapeutic agents is compromised the cause is missing or defective p53.

In endocytotic oxLDL induced cell death, components of the oxLDL destabilize the lysosomal membrane (LMP) leading to apoptosis. (See FIG. 1B.) The principles of these findings may be more broadly applied in developing a mechanism explaining the pathway by which the various therapies results in apoptosis.

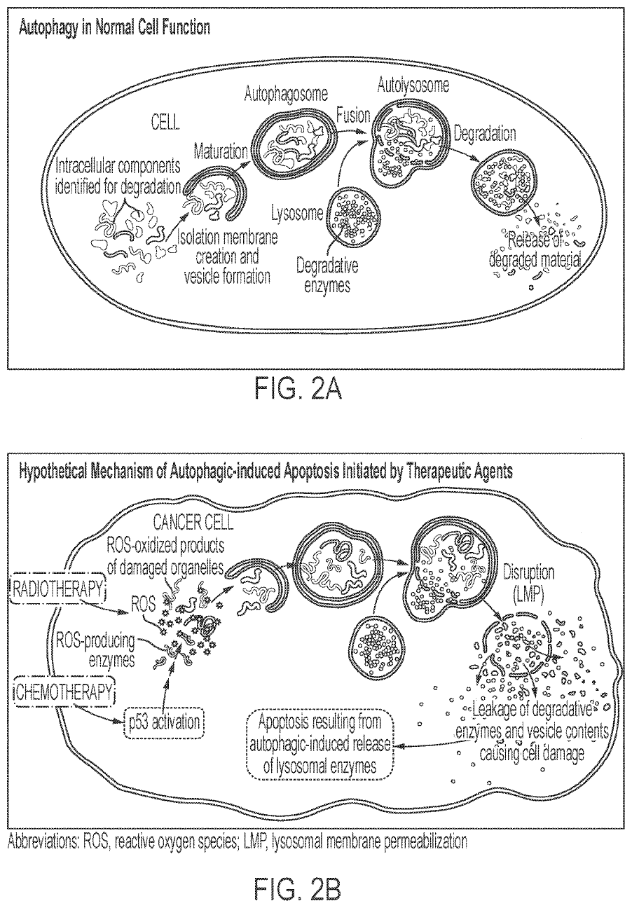

Phagasomes collect damaged or worn out cellular components which are incorporated into autophagasomes. These materials are then incorporated into autolysosomes. Under normal conditions, these collected components within the autolysosome are degraded and harmlessly and normally released into the cytoplasm. See FIG. 2A. In the cases of chemotherapeutic agents, radiation and perhaps other therapies as well, it is believed that the components that phagasomes gather up include products of ROS reaction with cellular components including cellular and organellar membranes. These ROS reaction products, similar to the oxidized components of LDL, then act to destabilize the autolysosomal membrane (LMP) resulting in release of degradative enzymes from the lysosome into the cytoplasm leading to autophagasomal lysosomal apoptosis. See FIG. 2B.

FIG. 2A shows normal function of autophagasomes. Cellular contents are brought into lysosomes which degrade and recycle the materials. FIG. 2B shows a mechanism of autophagic-induced apoptosis. Both radiotherapy and chemotherapy induce ROS which react with cellular contents including membranes of organelles. The oxidized materials, when internalized in lysosomes, may cause disruption of the lysosome membrane and leakage of the lysosomal degradative enzymes into the cytoplasm resulting in apoptosis in a manner similar to that shown in FIG. 1B.

Further focus on the role of oxidized lipids comes from preclinical and clinical reports on the effects of polyunsaturated fatty acids and statins on the efficacy of various cancer therapies. Increasing the content of polyunsaturated lipids both in circulating lipoproteins and in cellular membranes may facilitate the efficient operation of these mechanisms. It has been shown that inclusion of such lipids in diet of subject undergoing various cancer therapies influences the outcome of the therapies. Inclusion of such lipids in diet or as supplements prior to and during therapy is a relatively simple matter. They can be given orally with the result of enhancing their presence in both circulating lipoproteins, and cellular and organellar membranes. In cell culture, it has been shown that the activity of a variety of chemotherapeutic agents were enhanced by treatment of cells with n-3 fatty acids. These include 5-fluorouracil, doxorubicin, docetaxel, and cisplatin. Clinical trials of oral supplementation with n-3 polyunsaturated fatty acids during chemotherapy have shown that such supplementation improved chemotherapy efficacy in breast, colorectal, non-small cell lung, and pancreatic cancers. For example, breast cancer patients who had significant incorporation of n-3 fatty acids during treatment with cyclophosphamide, 5-fluorouracil and epirubicin experienced a longer time to disease progression and longer survival.

Pretreatment or concurrent treatment with statins has been shown to improve outcomes of a variety of therapies. For example, in patients with advanced hepatocellular carcinoma receiving 5-fluoroucacil those who received pravastatin as well had a median survival time of 18 months vs 9 months in the control group.

Focusing on the mechanism of ROS induced apoptosis, both extracellular such as the endocytotic uptake of oxLDL and intracellular such as the hypothesized autophagasome collection of oxidative debris and damaged subcellular constituents both leading to apoptotic cell death may translate into a framework for advancing cancer therapeutics. Focusing on the extracellular microenvironment of the tumor may lead to therapies which enhance localized production of ROS in that microenvironment resulting in endocytotosis initiated apoptosis. Focusing on intracellular production of ROS, including p53 mediated ROS production may also result in new ways to encourage autophagocytic apoptosis. These pathways may lead to potentially abundant production of neoantigens which activate ICD.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An article, comprising:
a complex comprising an antibody that recognizes a tumor, and an enzyme that is able to increase lysosomal membrane permeability.

2. (canceled)

3. The article of claim 1, wherein the enzyme is an oxidase.

4. The article of claim 3, wherein the oxidase is glucose oxidase.

5. The article of claim 3, wherein the oxidase is xanthine oxidase.

6. The article of claim 1, wherein the enzyme is a peroxidase.

7. The article of claim 1, wherein the antibody and the enzyme are directly covalently linked.

8. The article of claim 1, wherein the antibody and the enzyme are linked via a linking agent.

9-10. (canceled)

11. A method, comprising:
administering, to a subject, a composition comprising a complex of an antibody that recognizes a tumor, and an enzyme that is able to cause lysosomal leakage.

12. The method of claim 9, wherein the enzyme is an oxidase.

13. The method of claim 12, wherein the oxidase is glucose oxidase.

14. The method of claim 12, wherein the oxidase is xanthine oxidase.

15. The method of claim 9, wherein the enzyme is a peroxidase.

16. The method of claim 9, wherein the antibody and the enzyme are directly covalently linked.

17. The method of claim 9, wherein the antibody and the enzyme are linked via a linking agent.

18. The method of claim 9, further comprising administering a fatty acid to the subject.

19. The method of claim 18, wherein the fatty acid comprises an unsaturated C₃-C₆ fatty acid.

20. The method of claim 18, wherein the composition comprises the fatty acid.

21. The method of claim 18, wherein administering the complex and administering the fatty acid occur simultaneously.

22. The method of claim 18, wherein administering the complex and administering the fatty acid occur sequentially.

23. The method of claim 8, further comprising administering a statin to the subject.

24-26. (canceled)
